(19) United States
(12) Patent Application Publication
(10) Pub. No.: US 2025/0204835 A1
(43) Pub. Date: Jun. 26, 2025

(54) SYSTEM AND METHOD FOR ELECTROCARDIOGRAM (ECG) INTERPRETATION USING AN ARITIFICAL INTELLIGENCE (AI) MODEL AND A RULE-BASED ECG ANALYSIS MODEL

(71) Applicants: GE Precision Healthcare LLC, Waukesha, WI (US); Northwestern Memorial HealthCare, Chicago, IL (US)

(72) Inventors: Emmanouil Kargiantoulakis, Milwaukee, WI (US); Mozziyar Etemadi, Chicago, IL (US)

(21) Appl. No.: 18/988,374

(22) Filed: Dec. 19, 2024

Related U.S. Application Data
(60) Provisional application No. 63/622,765, filed on Jan. 19, 2024.

(30) Foreign Application Priority Data
Dec. 21, 2023 (GR) 20230101062

(51) Int. Cl. A61B 5/346; A61B 5/00; G16H 50/20

(52) U.S. Cl. CPC A61B 5/346; A61B 5/7267; G16H 50/20

(57) ABSTRACT

Various systems and methods are provided for ECG interpretation using an AI model and a rule-based ECG analysis model. An ECG may be received. A first ECG interpretation result of the ECG may be determined using an AI model. A second ECG interpretation result of the ECG may be determined using a rule-based ECG analysis model. A third ECG interpretation result of the ECG may be determined based on the first ECG interpretation result and the second ECG interpretation result. The third ECG interpretation result may be provided.

### CROSS-REFERENCE TO RELATED APPLICATIONS

The present application claims priority to U.S. Provisional Application No. 63/622,765, filed on Jan. 19, 2024, and Greek Application No. 20230101062, filed on Dec. 21, 2023, the entire contents of which is hereby incorporated by reference.

### BACKGROUND

An electrocardiogram (ECG) is a graphic representation of electrical activity of the heart, and is generally represented as a waveform. A rule-based ECG analysis model may receive an ECG, determine a set of features of the ECG, and generate a diagnostic interpretation result of the ECG based on the set of features. A physician may review the ECG and/or the diagnostic interpretation result to assess the cardiac activity of a patient.

### SUMMARY

This summary introduces concepts that are described in more detail in the detailed description. It should not be used to identify essential features of the claimed subject matter, nor to limit the scope of the claimed subject matter.

In an aspect, a method may include receiving an electrocardiogram (ECG); determining a first ECG interpretation result of the ECG using an artificial intelligence (AI) model; determining a second ECG interpretation result of the ECG using a rule-based ECG analysis model; determining a third ECG interpretation result of the ECG based on the first ECG interpretation result and the second ECG interpretation result; and providing the third ECG interpretation result.

In another aspect, a device may include a memory storing instructions; and one or more processors configured to execute the instructions to: receive an electrocardiogram (ECG); determine a first ECG interpretation result of the ECG using an artificial intelligence (AI) model; determine a second ECG interpretation result of the ECG using a rule-based ECG analysis model; determine a third ECG interpretation result of the ECG based on the first ECG interpretation result and the second ECG interpretation result; and provide the third ECG interpretation result

In yet another aspect, a non-transitory computer-readable medium may store instructions that, when executed by one or more processors, cause the one or more processors to: receive an electrocardiogram (ECG); determine a first ECG interpretation result of the ECG using an artificial intelligence (AI) model; determine a second ECG interpretation result of the ECG using a rule-based ECG analysis model determine a third ECG interpretation result of the ECG based on the first ECG interpretation result and the second ECG interpretation.

### BRIEF DESCRIPTION OF DRAWINGS

FIG. 1 is a diagram of an example system for ECG interpretation using an AI model and a rule-based ECG analysis model.

FIG. 2 is a diagram of example components of a device of the example system shown in FIG. 1.

FIG. 3 is a diagram of an example process of using an AI model.

FIG. 4 is a diagram of an example process for training of an AI model.

FIG. 5 is a diagram of an example process for ECG interpretation using an AI model and a rule-based ECG analysis model.

FIG. 6 is a flowchart of an example process for ECG interpretation using an AI model and a rule-based ECG analysis model.

FIGS. 7A-7C are diagrams of an example process for ECG interpretation using an AI model and a rule-based ECG analysis model.

FIG. 8 is a diagram of an example process for training an AI model.

### DETAILED DESCRIPTION

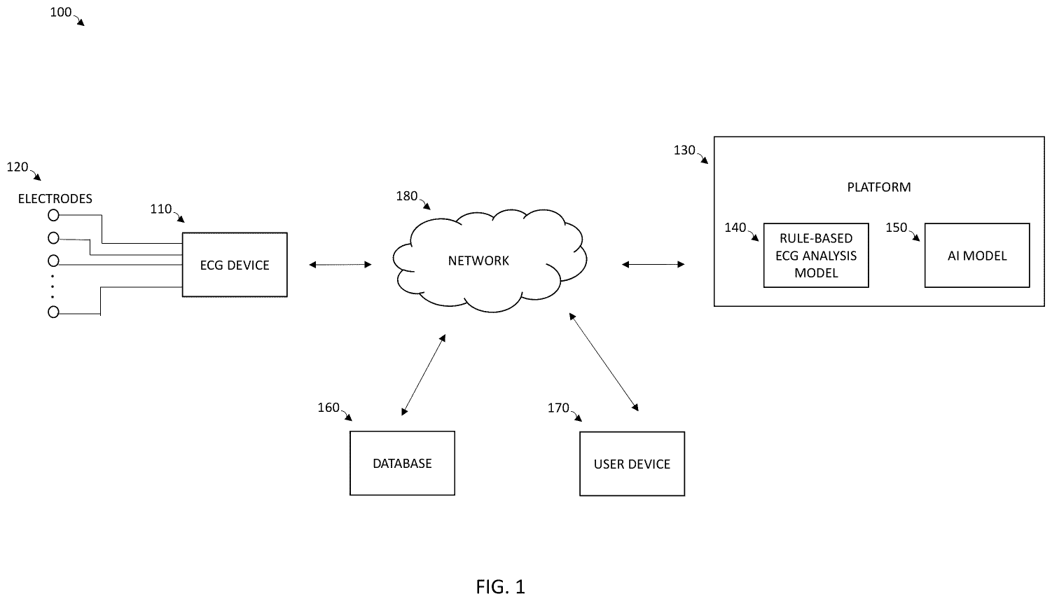

FIG. 1 is a diagram of an example system 100 for ECG interpretation using an AI model and a rule-based ECG analysis model. As shown in FIG. 1, the system 100 may include an ECG device 110, electrodes 120, a platform 130, a rule-based ECG analysis model 140, an AI model 150, a database 160, a user device 170, and a network 180.

The ECG device 110 may be configured to generate an ECG of a patient. For example, the ECG device 110 may be a stand-alone ECG device, a portable ECG device, a multi vital sign monitoring device, or the like. The ECG device 110 may receive cardiac electrical signals via the electrodes 120, and generate the ECG based on the cardiac electrical signals. The ECG may be a 3-lead ECG, a 5-lead ECG, a 6-lead ECG, a 12-lead ECG, or the like. The ECG device 110 may include any number of electrodes 120. For example, the ECG device 110 may include ten electrodes 120 for generating a 12-lead ECG.

The platform 130 may be configured to receive an ECG from the ECG device 110, determine a first ECG interpretation result using the rule-based ECG analysis model 140, determine a second ECG interpretation result using the AI model 150, and determine a third ECG interpretation result based on the first ECG interpretation result and the second ECG interpretation result. For example, the platform 130 may be a server, a cloud computing system, or the like.

The rule-based ECG analysis model 140 may be configured to receive an ECG from the ECG device 110, determine a set of features of the ECG, and determine an ECG interpretation result of the ECG. For example, the rule-based ECG analysis model 140 may be the Marquette™ 12SL ECG analysis program, or the like.

The AI model 150 may be configured to receive an ECG from the ECG device 110, and determine an ECG interpretation result based on the ECG. For example, the AI model 150 may be a transformer model, a decision tree, a linear regression model, a neural network (e.g., a deep neural network (DNN), a convolutional neural network (CNN), a recurrent neural network (RNN), or the like), a logistic regression model, a support vector machine, or the like.

The database 160 may be configured to store an ECG, an ECG interpretation result of the ECG, or the like. For example, the database 160 may be a hierarchical database, a network database, a relational database, or the like.

The user device 170 may be configured to receive an ECG interpretation result from the platform 130, and provide the diagnostic interpretation result for display. For example, the user device 170 may be a smartphone, a laptop computer, a desktop computer, a wearable device, a medical device, or the like.

The network 180 may be configured to permit communication between the devices of the system 100. For example, the network 180 may be a cellular network (e.g., a fifth generation (5G) network, a long-term evolution (LTE) network, a third generation (3G) network, a code division multiple access (CDMA) network, etc.), a public land mobile network (PLMN), a local area network (LAN), a wide area network (WAN), a metropolitan area network (MAN), a telephone network (e.g., the Public Switched Telephone Network (PSTN)), a private network, an ad hoc network, an intranet, the Internet, a fiber optic-based network, or the like, and/or a combination of these or other types of networks.

The platform 130 may provide the AI model 150 (e.g., a transformer neural network for full ECG interpretation), which is trained on ECG interpretations (e.g., 12SL statements) as its “vocabulary,” that is, as discretized semantic tokens. Then, the ECG interpretation may be given as next-token prediction resulting in a string of statements, which may be the same format the full interpretation is provided by the rule-based ECG analysis model (e.g., 12SL), even after edits by over-readers. The formulation with the 12SL tokenization may be much more stable than just next-word prediction. These embodiments may also uniquely allow a hybrid integration with the rule-based ECG analysis model 140 (e.g., the 12SL algorithm), which would imbue the full hybrid model with the robustness and interpretability of the rule-based ECG analysis model (e.g., 12SL). The flexibility of the AI model 150 (e.g., the transformer) input may provide a much more seamless way to provide more information from the same patient, such as previous ECGs. Finally, the rule-based ECG analysis model 140 (e.g., 12SL) may provide a uniquely well-suited statement library for diagnostic tokens, due to the databases 160 that may use this statement library and may be leveraged for training. In this way, some embodiments provide more powerful and accurate ECG interpretation, resulting in a reduced need and time spent from physicians to edit, as well as improved patient outcomes.

The number and arrangement of the devices of the system 100 shown in FIG. 1 are provided as an example. In practice, the system 100 may include additional devices, fewer devices, different devices, or differently arranged devices than those shown in FIG. 1. Additionally, or alternatively, a set of devices (e.g., one or more devices) of the system 100 may perform one or more functions described as being performed by another set of devices of the system 100.

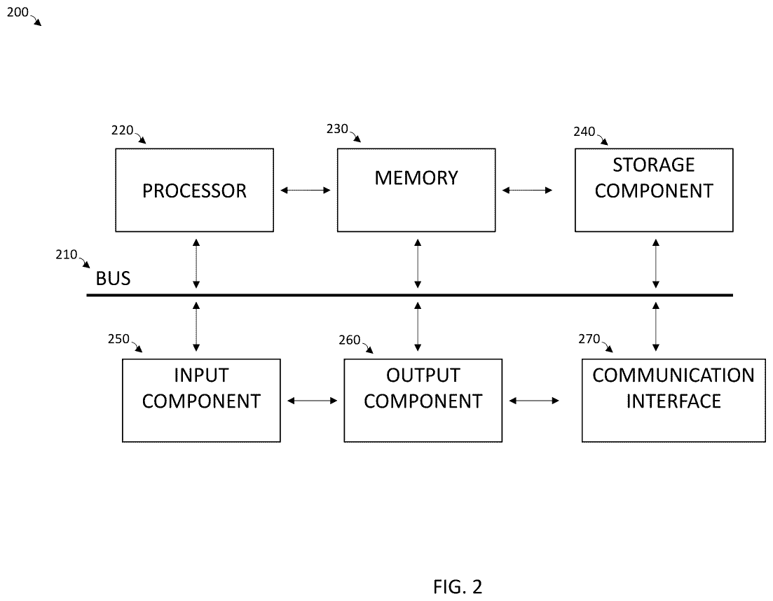

FIG. 2 is a diagram of example components of a device 200 of the example system shown in FIG. 1. The device 200 may correspond to the ECG device 110, the platform 130, the database 160 and/or the user device 170. As shown in FIG. 2, the device 200 may include a bus 210, a processor 220, a memory 230, a storage component 240, an input component 250, an output component 260, and a communication interface 270.

The bus 210 includes a component that permits communication among the components of the device 200. The processor 220 may be implemented in hardware, firmware, or a combination of hardware and software. The processor 220 may be a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component. The processor 220 may include one or more processors capable of being programmed to perform a function. The processor 220 may include one or more processors 220 configured to perform the operations described herein. For example, a single processor 220 may be configured to perform all of the operations described herein. Alternatively, multiple processors 220, collectively, may be configured to perform all of the operations described herein, and each of the multiple processors 220 may be configured to perform a subset of the operations descried herein. For example, a first processor 220 may perform a first subset of the operations described herein, a second processor 220 may be configured to perform a second subset of the operations described herein, etc.

The memory 230 may include a random access memory (RAM), a read only memory (ROM), and/or another type of dynamic or static storage device (e.g., a flash memory, a magnetic memory, and/or an optical memory) that stores information and/or instructions for use by the processor 220.

The storage component 240 may store information and/or software related to the operation and use of the device 200. For example, the storage component 240 may include a hard disk (e.g., a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive.

The input component 250 may include a component that permits the device 200 to receive information, such as via user input (e.g., a touch screen display, a keyboard, a keypad, a mouse, a button, a switch, a camera, and/or a microphone for receiving the reference audio input and/or visual input). Additionally, or alternatively, the input component 250 may include a sensor for sensing information (e.g., a global positioning system (GPS) component, an accelerometer, a gyroscope, and/or an actuator). The output component 260 may include a component that provides output information from the device 200 (e.g., a display, a speaker for outputting sound at the output sound level, and/or one or more light-emitting diodes (LEDs)).

The communication interface 270 may include a transceiver-like component (e.g., a transceiver and/or a separate receiver and transmitter) that enables the device 200 to communicate with other devices, such as via a wired connection, a wireless connection, or a combination of wired and wireless connections. The communication interface 270 may permit the device 200 to receive information from another device and/or transmit information to another device. For example, the communication interface 270 may include an Ethernet interface, an optical interface, a coaxial interface, an infrared interface, a radio frequency (RF) interface, a universal serial bus (USB) interface, a Wi-Fi interface, a cellular network interface, or the like.

The device 200 may perform one or more processes described herein. The device 200 may perform these processes based on the processor 220 executing software instructions stored by a non-transitory computer-readable medium, such as the memory 230 and/or the storage component 240. A computer-readable medium may be defined herein as a non-transitory memory device. A memory device may include memory space within a single physical storage device or memory space spread across multiple physical storage devices.

The software instructions may be read into the memory 230 and/or the storage component 240 from another computer-readable medium or from another device via the communication interface 270. When executed, the software instructions stored in the memory 230 and/or the storage component 240 may cause the processor 220 to perform one or more processes described herein. Additionally, or alternatively, hardwired circuitry may be used in place of or in combination with software instructions to perform one or more processes described herein. Thus, implementations described herein are not limited to any specific combination of hardware circuitry and software.

The number and arrangement of the components shown in FIG. 2 are provided as an example. In practice, the device 200 may include additional components, fewer components, different components, or differently arranged components than those shown in FIG. 2. Additionally, or alternatively, a set of components (e.g., one or more components) of the device 200 may perform one or more functions described as being performed by another set of components of the device 200.

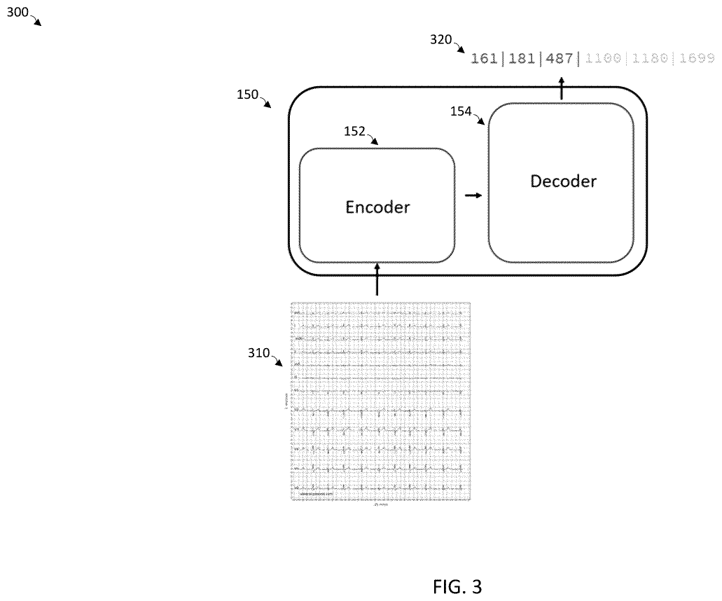

FIG. 3 is a diagram of an example process 300 of using an AI model. As shown in FIG. 3, the AI model 150 may include an encoder 152 and a decoder 154. The encoder 152 may receive an ECG 310. The decoder may output an ECG interpretation result 320.

The rule-based ECG analysis model 140 may output an ECG interpretation result in the form of a string of statement identifiers. For example, the ECG interpretation result may be “161|181|487|1100|1180|1699.” Each of these statement identifiers may represent a diagnostic statement from a library (e.g., the 12SL library). Even edits by over-readers may mostly maintain that statement library. According to an embodiment, fully human text may be added outside that library. According to an embodiment, those ECGs may be excluded from training. By treating ECG interpretation results (e.g., diagnostic statements) as semantic tokens, the AI model 150 (e.g., the transformer) may be trained to encode embeddings that carry diagnostic information towards ECG interpretation. The embedded space may be a more complete encoding of the ECG signal than the tailored features extracted from the rule-based ECG analysis model 140 (e.g., 12SL). According to an embodiment, self-supervised training may be performed separately from the target diagnostic output to optimize, or at least improve, deep feature extraction.

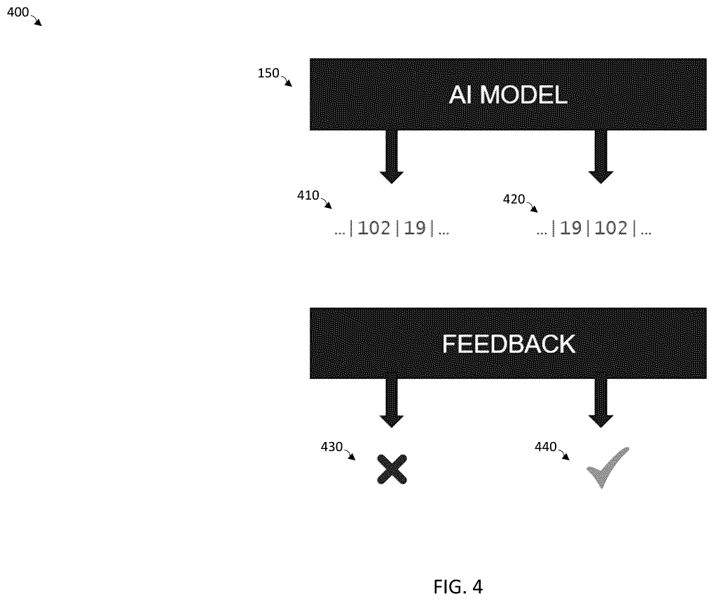

FIG. 4 is a diagram of an example process 400 for training of an AI model. As shown in FIG. 4, the AI model 150 may output an ECG interpretation result 410 having a sequence of diagnostic statements of “102” and “19,” and may output an ECG interpretation result 420 having a sequence of “19” and “102.” As shown by reference number 430, the sequence of the ECG interpretation result 410 may be rejected. As shown by reference number 440, the sequence of the ECG interpretation result 420 may be accepted. For instance, statement “102” (“with short PR”) is a modifier to statement “19” (“Sinus rhythm”) and should only appear after it. The reverse might be meaningless. Several such “rules” exist in the library of the rule-based ECG analysis model 140 (e.g., the 12SL statement library) and define which output is meaningful.

Transformers used for next-word prediction may form the basis of Large-Language Models (LLMs). LLMs may suffer from instabilities, sometimes extreme enough to be called “hallucinations.” Embodiments herein may use a library of the rule-based ECG analysis model 140 (e.g., the 12SL statement library) which may be more robust. For LLMs, a component for stabilization may be reinforcement learning through human feedback (RLHF). For diagnostic cardiology, this may be infeasible due to the high level of expertise needed to provide that feedback. But, when using the library of the rule-based ECG analysis model 140 (e.g., the 12SL statement library), this feedback may be achieved with hard rules from the vocabulary, which may practically eliminate “unphysical” hallucinations. In this way, the powerful architecture is coupled with a very stable output vocabulary, which permits a correct diagnosis to be learnt in a supervised manner.

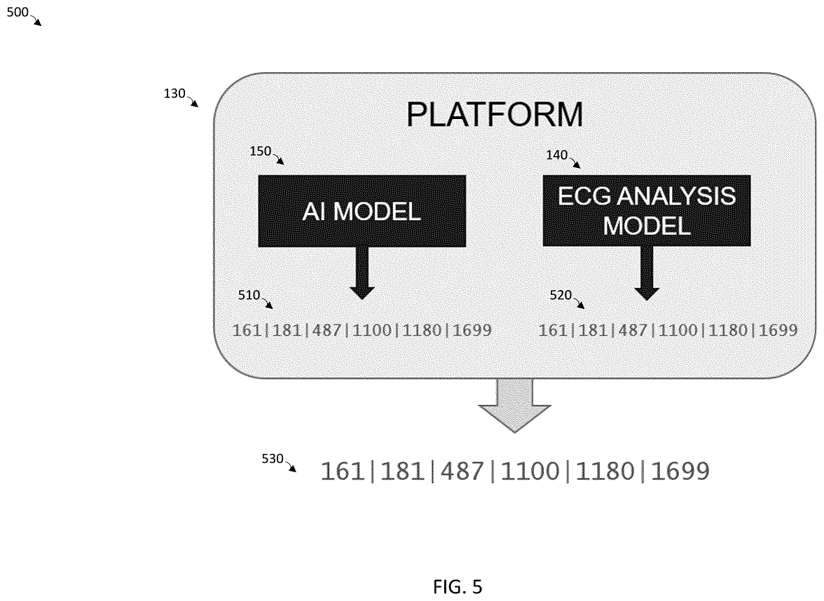

FIG. 5 is a diagram of an example process 500 for ECG interpretation using an AI model and a rule-based ECG analysis model. As shown in FIG. 5, the platform 130 may implement the rule-based ECG analysis model 140 and the AI model 150. The AI model 150 may output an ECG interpretation result 510, and the rule-based ECG analysis model 140 may output an ECG interpretation result 520. The platform 130 may determine an ECG interpretation result 530 based on the ECG interpretation result 510 and the ECG interpretation result 520.

For an AI model 150 (e.g., a transformer) implementing an untethered next-word prediction, it might be difficult to even evaluate its performance on a database 160 of validated ECGs, especially on separate diagnostic endpoints as might be required by the FDA. Based on the output being given as ECG interpretation results (e.g., 12SL statements), performance evaluation may become trivial. Some embodiments may also allow integration with the rule-based ECG analysis model 140 in a hybrid system. The rule-based ECG analysis model 140 (e.g., 12SL) may have full explainability, and, at the very least, may provide significant context for the final output. It may be robust and operate with high specificity, grounding the system even more; and is trusted by physicians globally. In contrast, LLMs might be relatively unfit for clinical practice. The hybrid integration may include the AI model 150 (e.g., the transformer) output triggering new decision paths of the rule-based ECG analysis model 140. Alternatively, the hybrid integration could take the form of a stacking ensemble optimized in a data-driven manner. As a “mixture of experts,” this embodiment may also allow estimation of confidence in the diagnostic output.

Some embodiments herein may use 12SL statements as diagnostic tokens for a transformer model for ECG interpretation. Further, some embodiments herein provide a powerful synergy and complementarity from the integration of 12SL with a deep neural network into a hybrid system, such as in the form of a mixture of experts.

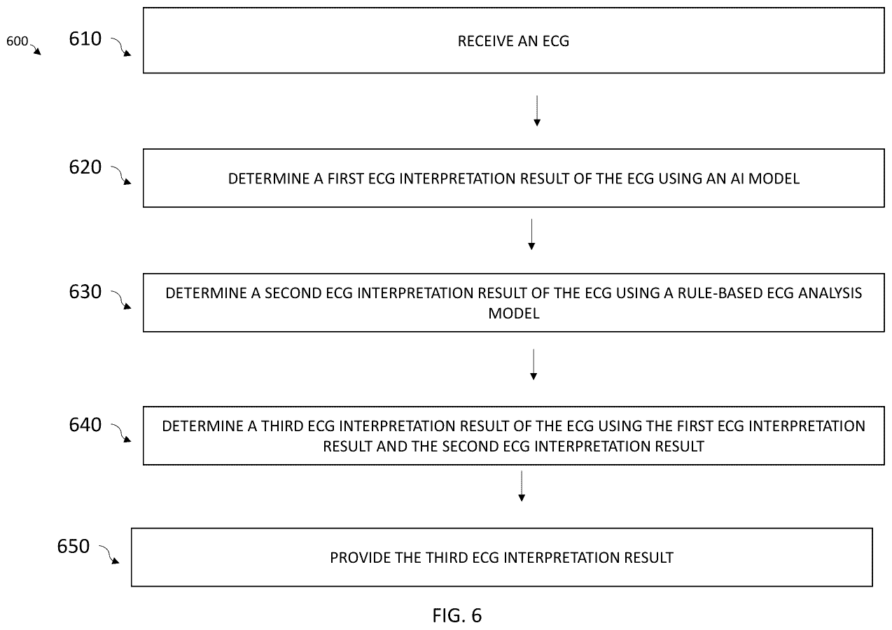

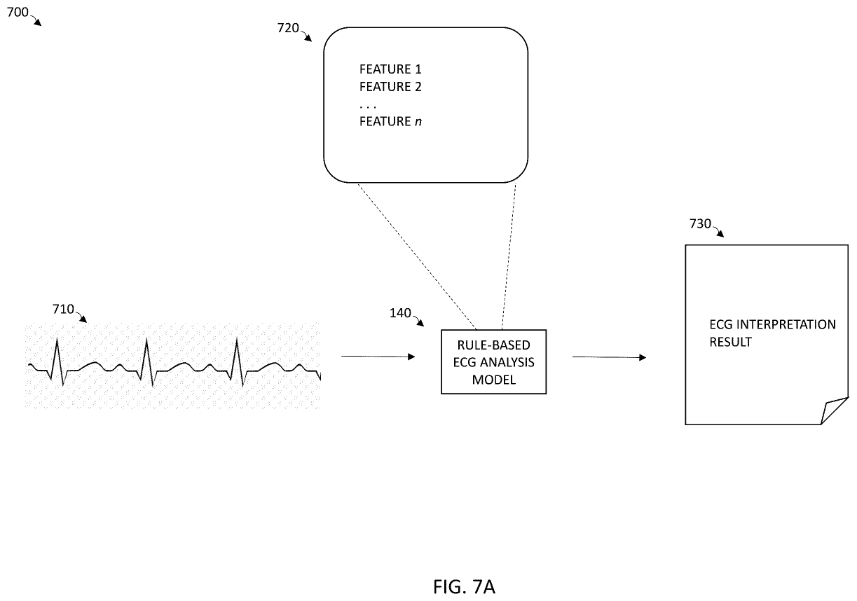

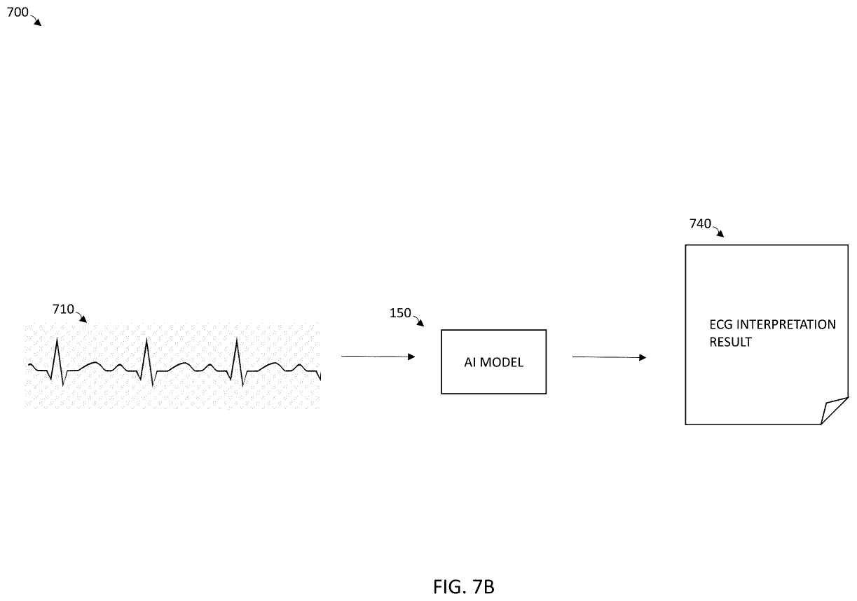

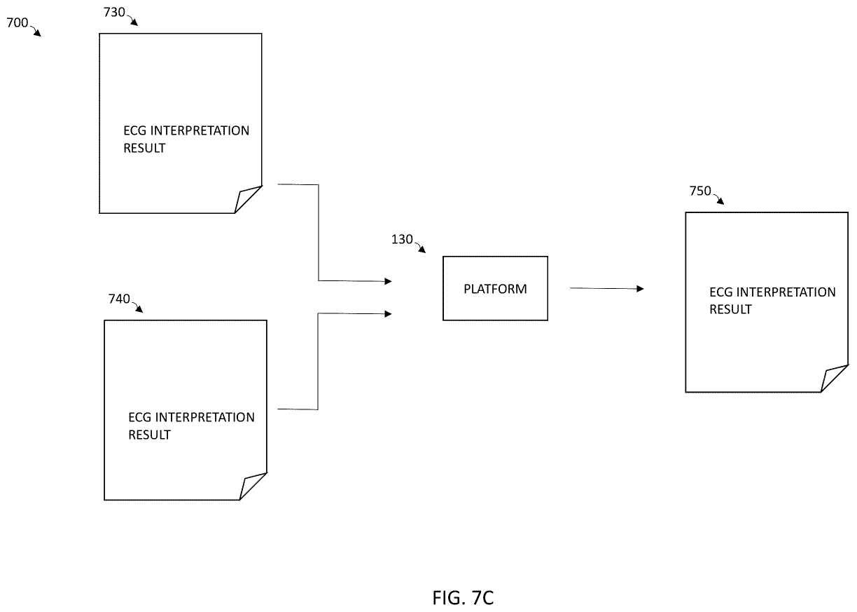

FIG. 6 is a flowchart of an example process 600 for ECG interpretation using an AI model and a rule-based ECG analysis model. FIGS. 7A-7C are diagrams of an example process 700 for ECG interpretation using an AI model and a rule-based ECG analysis model.

As shown in FIG. 6, the process 600 may include receiving an ECG (operation 610). For example, the platform 130 may receive an ECG 710. As further shown in FIG. 6, the process 600 may include determining a first ECG interpretation result of the ECG using an AI model (operation 620). The first ECG interpretation result may include a diagnosis of the ECG. For example, the first ECG interpretation result may include a diagnosis, such as atrial-paced rhythm, ventricular-paced rhythm, atrial flutter, ectopic atrial tachycardia, sinus bradycardia, sinus tachycardia, junctional bradycardia, atrial fibrillation, left bundle branch block, septal infarct, or the like.

As shown in FIG. 7A, the platform 130 may input the ECG 710 into the rule-based ECG analysis model 140. The rule-based ECG analysis model 140 may determine features 720 of the ECG 710, and determine an ECG interpretation result 730 based on the features 720 of the ECG 710. The set of features of the ECG determined by the rule-based ECG analysis model 140 may include an amplitude of a wave of the ECG, a duration of a wave of the ECG, or the like. For example, the set of features may include an amplitude of a P wave, a duration of the P wave, an amplitude of a Q wave, a duration of the Q wave, an amplitude of an R wave, a duration of the R wave, a PR interval, an amplitude of an S wave, a duration of the S wave, a duration of the QRS complex, an amplitude of a T wave, a duration of the T wave, a QT interval, or the like. For example, as shown in FIG. 7A, the rule-based ECG analysis model 140 may receive an ECG 710, and determine a set of features 720 of the ECG 710 (e.g., feature 1, feature 2, . . . , feature n, etc.).

As further shown in FIG. 6, the process 600 may include determining a second ECG interpretation result of the ECG using a rule-based ECG analysis model (operation 630). The second ECG interpretation result may include a diagnosis of the ECG. For example, the second ECG interpretation result may include a diagnosis, such as atrial-paced rhythm, ventricular-paced rhythm, atrial flutter, ectopic atrial tachycardia, sinus bradycardia, sinus tachycardia, junctional bradycardia, atrial fibrillation, left bundle branch block, septal infarct, or the like. As shown in FIG. 7B, the platform 130 may input the ECG 710 into the AI model 150. The AI model 150 may determine an ECG interpretation result 740 based on the ECG 710.

As further shown in FIG. 6, the process 600 may include determining a third ECG interpretation result of the ECG using the first ECG interpretation result and the second ECG interpretation result (operation 640). For example, the platform 130 may determine an ECG interpretation result based on the ECG interpretation results.

According to an embodiment, the platform 130 may determine the ECG interpretation result which is the same as the ECG interpretation result of the rule-based ECG analysis model 140. That is, the platform 130 may select the ECG interpretation result of the rule-based ECG analysis model 140 as the ECG interpretation result. Alternatively, the platform 130 may determine the ECG interpretation result which is the same as the ECG interpretation result of the AI model 150. That is, the platform 130 may select the ECG interpretation result of the AI model 150 as the ECG interpretation result. Alternatively, the platform 130 may determine an ECG interpretation result that is a combination of the ECG interpretation result of the rule-based ECG analysis model 140 an the AI model 150. For example, the platform 130 may weigh the ECG interpretation results, assign scores to the ECG interpretation results, or the like, and determine an ECG interpretation result that is some combination or permutation of the ECG interpretation results.

As an example, and as shown in FIG. 7C, the platform 130 may determine the ECG interpretation result 750 based on the ECG interpretation result 730 and the ECG interpretation result 740.

As further shown in FIG. 6, the process 600 may include providing the third ECG interpretation result (operation 650). For example, the platform 130 may provide the ECG interpretation result for display, to another device (e.g., the user device 170 for display), or the like.

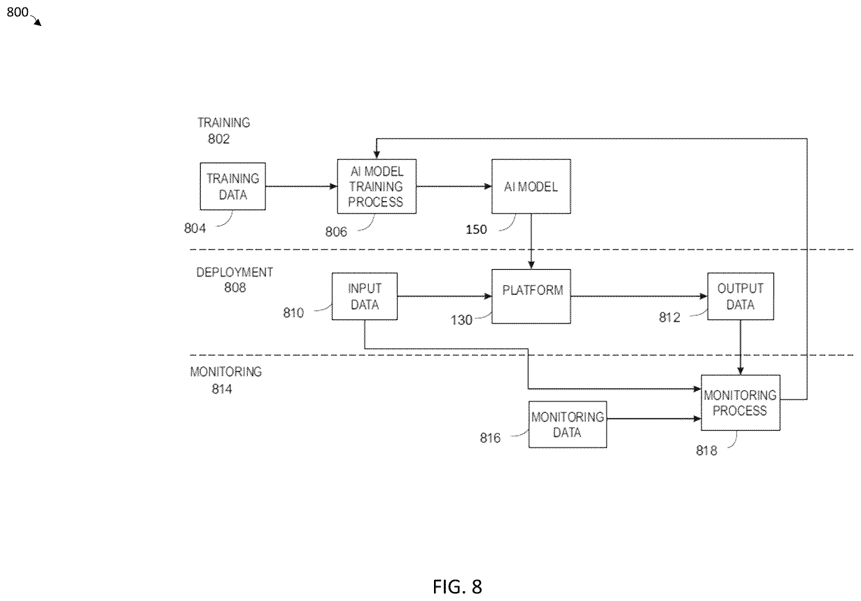

FIG. 8 is a diagram of an example process 800 for training an AI model. The platform 130 may generate, store, train, and/or use the AI model 150. According to an embodiment, the platform 130 may include the AI model 150 and/or instructions associated with the AI model 150. For example, the platform 130 may include instructions for generating the AI model 150, training the AI model 150, using the AI model 150, etc. According to another embodiment, a system or device other than the platform 130 may be used to generate and/or train the AI model 150. For example, a system or device may include instructions for generating the AI model 150, and/or instructions for training the AI model 150. The system or device may provide a resulting trained AI model 150 to the platform 130 for use.

As shown in FIG. 8, according to an embodiment, the process 800 may include a training phase 802, a deployment phase 808, and a monitoring phase 814. In the training phase 802, at operation 806, the process 1100 may include receiving and processing training data 804 to generate a trained AI model 150 for determining an ECG interpretation result. The training data 804 may be generated, received, or otherwise obtained from internal and/or external resources. The training data may include 12SL statements as diagnostic tokens.

Generally, the AI model 150 may include a set of variables (e.g., nodes, neurons, filters, or the like) that are tuned (e.g., weighted, biased, or the like) to different values via the application of the training data 804. According to an embodiment, the training process at operation 806 may employ supervised, unsupervised, semi-supervised, and/or reinforcement learning processes to train the AI model 150. According to an embodiment, a portion of the training data 804 may be withheld during training and/or used to validate the trained AI model 150.

For supervised learning processes, the training data 804 may include labels or scores that may facilitate the training process by providing a ground truth. For example, the labels or scores may indicate an ECG interpretation result. Training may proceed by feeding a training dataset including the training data 804 into the AI model 150. The AI model 150 may have variables set at initialized values (e.g., at random, based on Gaussian noise, based on pre-trained values, or the like). The AI model 150 may output an ECG interpretation result based on the ECG being input to the AI model 150. The output may be compared with the corresponding label or score (e.g., the ground truth) indicating the ECG interpretation result, which may then be back-propagated through the AI model 150 to adjust the values of the variables. This process may be repeated for a plurality of samples at least until a determined loss or error is below a predefined threshold. According to an embodiment, some of the training data 804 may be withheld and used to further validate or test the trained AI model 150.

For unsupervised learning processes, the training data 804 may not include pre-assigned labels or scores to aid the learning process. Instead, unsupervised learning processes may include clustering, classification, or the like, to identify naturally occurring patterns in the training data 804. As an example, the training data may be clustered into groups based on identified similarities and/or patterns. K-means clustering or K-Nearest Neighbors may also be used, which may be supervised or unsupervised. Combinations of K-Nearest Neighbors and an unsupervised cluster technique may also be used. For semi-supervised learning, a combination of training data 804 with pre-assigned labels or scores and training data 804 without pre-assigned labels or scores may be used to train the AI model 150.

When reinforcement learning is employed, an agent (e.g., an algorithm) may be trained to make a decision regarding the ECG interpretation result from the training data 804 through trial and error. For example, based on making a decision, the agent may then receive feedback (e.g., a positive reward if the prediction was above a predetermined threshold), adjust its next decision to maximize the reward, and repeat until a loss function is optimized.

After being trained, the trained AI model 150 may be stored and subsequently applied by the platform 130 during the deployment phase 808. For example, during the deployment phase 808, the trained AI model 150 executed by the platform 130 may receive input data 810. The input data 810 may include an ECG. The AI model 150 may provide, as output data 812, an ECG interpretation result of the ECG.

The monitoring data 816 may include data that identifies an ECG interpretation result as determined by an operator. During process 818, the monitoring data 816 may be analyzed along with the predicted output data 812 and input data 810 to determine an accuracy of the trained AI model 150. According to an embodiment, based on the analysis, the process 800 may return to the training phase 802, where at operation 806 values of one or more variables of the model may be adjusted to improve the accuracy of the AI model 150.

The example process 800 described above is provided merely as an example, and may include additional, fewer, different, or differently arranged aspects than depicted in FIG. 8.

FIG. 8 describes the training, deployment, and monitoring associated with a trained AI model 150 for determining an ECG interpretation result. According to an embodiment, one or more other trained AI model 150s may be applied. Each of the trained AI model 150s may include similar training, deployment, and/or monitoring phases as described above for the trained AI model 150 in FIG. 8, however the particular types of training data, input data, output data, and monitoring data may be different.

Although the embodiments herein are associated with ECGs and a rule-based ECG analysis model, it should be understood that the embodiments herein are applicable to other types of biosignals and other types of rule-based biosignal analysis models. For example, the embodiments herein are applicable to electroencephalograms (EEGs), electromyograms (EMGs), electrooculograms (EOGs), electroretinograms (ERGs), electrogastrograms (EGGs), or the like, and respective rule-based analysis models that are associated with the foregoing biosignals.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

What is claimed is:

1. A method comprising:
receiving an electrocardiogram (ECG);
determining a first ECG interpretation result of the ECG using an artificial intelligence (AI) model;
determining a second ECG interpretation result of the ECG using a rule-based ECG analysis model;
determining a third ECG interpretation result of the ECG based on the first ECG interpretation result and the second ECG interpretation result; and
providing the third ECG interpretation result.

2. The method of claim 1, wherein the first ECG interpretation result, the second ECG interpretation result, and the third ECG interpretation result define strings of statement identifies representing a diagnostic statement from a library.

3. The method of claim 1, wherein the first ECG interpretation result and the second ECG interpretation result include a diagnosis of atrial-paced rhythm, ventricular-paced rhythm, atrial flutter, ectopic atrial tachycardia, sinus bradycardia, sinus tachycardia, junctional bradycardia, atrial fibrillation, left bundle branch block, or septal infarct.

4. The method of claim 1, further comprising determining features of the ECG using the rule-based ECG model, and wherein the features are used to determine the second ECG model, and wherein the features include an amplitude of a wave of the ECG or a duration of a wave of the ECG.

5. The method of claim 1, wherein the AI model is trained on training data, and wherein the AI model includes a set of variables that are tuned to different values with the application of the training data.

6. The method of claim 5, wherein the training data includes scores that facilitate the training process by providing a ground truth, and wherein the first ECG interpretation result is compared with the corresponding score and back-propagated through the AI model to adjust the set of variables.

7. The method of claim 5, wherein the training data is clustered into groups based on identified similarities and patterns.

8. A device comprising:
a memory storing instructions; and
one or more processors configured to execute the instructions to:
receive an electrocardiogram (ECG);
determine a first ECG interpretation result of the ECG using an artificial intelligence (AI) model;
determine a second ECG interpretation result of the ECG using a rule-based ECG analysis model;
determine a third ECG interpretation result of the ECG based on the first ECG interpretation result and the second ECG interpretation result; and
provide the third ECG interpretation result.

9. The system of claim 9, wherein the first ECG interpretation result, the second ECG interpretation result, and the third ECG interpretation result define strings of statement identifies representing a diagnostic statement from a library.

10. The system of claim 9, wherein the first ECG interpretation result and the second ECG interpretation result include a diagnosis of atrial-paced rhythm, ventricular-paced rhythm, atrial flutter, ectopic atrial tachycardia, sinus bradycardia, sinus tachycardia, junctional bradycardia, atrial fibrillation, left bundle branch block, or septal infarct.

11. The system of claim 9, wherein the one or processors is also configured to execute instructions to determine features of the ECG using the rule-based ECG model, and wherein the features are used to determine the second ECG model, and wherein the features include an amplitude of a wave of the ECG or a duration of a wave of the ECG.

12. The system of claim 9, wherein the AI model is trained on training data, and wherein the AI model includes a set of variables that are tuned to different values with the application of the training data.

13. The system of claim 12, wherein the training data includes scores that facilitate the training process by providing a ground truth, and wherein the first ECG interpretation result is compared with the corresponding score and back-propagated through the AI model to adjust the set of variables.

14. The method of claim 12, wherein the training data is clustered into groups based on identified similarities and patterns.

15. A non-transitory computer-readable medium storing instructions that, when executed by one or more processors, cause the one or more processors to:
receive an electrocardiogram (ECG);
determine a first ECG interpretation result of the ECG using an artificial intelligence (AI) model;
determine a second ECG interpretation result of the ECG using a rule-based ECG analysis model;
determine a third ECG interpretation result of the ECG based on the first ECG interpretation result and the second ECG interpretation result; and
provide the third ECG interpretation result.

16. The non-transitory computer-readable medium of claim 15, wherein the first ECG interpretation result, the second ECG interpretation result, and the third ECG interpretation result define strings of statement identifies representing a diagnostic statement from a library.

17. The non-transitory computer-readable medium of claim 15, wherein the first ECG interpretation result and the second ECG interpretation result include a diagnosis of atrial-paced rhythm, ventricular-paced rhythm, atrial flutter, ectopic atrial tachycardia, sinus bradycardia, sinus tachycardia, junctional bradycardia, atrial fibrillation, left bundle branch block, or septal infarct.

18. The non-transitory computer-readable medium of claim 15, further comprising determining features of the ECG using the rule-based ECG model, and wherein the features are used to determine the second ECG model, and wherein the features include an amplitude of a wave of the ECG or a duration of a wave of the ECG.

19. The non-transitory computer-readable medium of claim 15, wherein the AI model is trained on training data, and wherein the AI model includes a set of variables that are tuned to different values with the application of the training data.

20. The non-transitory computer-readable medium of claim 19, wherein the training data includes scores that facilitate the training process by providing a ground truth, and wherein the first ECG interpretation result is compared with the corresponding score and back-propagated through the AI model to adjust the set of variables.
